(19) United States
(12) Patent Application Publication
(10) Pub. No.: US 2005/0187532 A1
(43) Pub. Date: Aug. 25, 2005

(54) Diaphragm-based reservoir for a closed blood sampling system

(75) Inventors: Bernd Thurau, Hoslwang (DE); Robert Herz, Rohrdorf (DE); Angela Crall, Columbus, OH (US); Dale Gallogly, Mount Vernon, OH (US)

Correspondence Address: WOOD, HERRON & EVANS, LLP, 2700 CAREW TOWER, 441 VINE STREET, CINCINNATI, OH, 45202, US

(73) Assignee: Medex, Inc., Carlsbad, CA (US)

(21) Appl. No.: 10/785,550

(22) Filed: Feb. 24, 2004

(51) Int. Cl. H01H 9/00; A61B 19/00; A61B 5/00; B65D 33/00; B65D 81/00

(52) U.S. Cl. 604/403; 604/407; 600/580

(57) ABSTRACT

A reservoir (10, 26, 80, 126, 226, 380) includes a rigid wall (12, 28, 82, 128, 228, 382), a flexible membrane (14, 34, 134, 234, 334) sealingly secured to the rigid wall (12, 28, 82, 128, 228, 382) to define a variable volume chamber (18, 38, 138, 238, 338), and inlet (40, 140, 240, 340) and exit (42, 142, 242, 342) ports in fluid communication with the chamber (18, 38, 138, 238, 338). A drive surface (24, 50, 150, 250, 350) engages the membrane (14, 34, 134, 234, 334) to position it adjacent the rigid wall (12, 28, 82, 128, 228, 382) to define a minimum volume position. The drive surface (24, 50, 150, 250, 350) may be moved so that the membrane (14, 34, 134, 234, 334) flexes out of the minimum volume position to an expanded volume position. The membrane (14, 34, 134, 234, 334) may be coupled to the drive surface (24, 50, 150, 250, 350) or be uncoupled from the drive surface (24, 50, 150, 250, 350). The reservoir (10, 26, 80, 126, 226, 326) may be included in a closed blood sampling system (88) for drawing a sample of whole blood.

### FIELD OF THE INVENTION

The present invention relates to closed blood sampling systems through which blood may be drawn from a patient, and more specifically, to an improved fluid storage device useful in facilitating the drawing of whole blood from a patient.

### BACKGROUND OF THE INVENTION

In certain medical situations, such as coronary intensive care, highly accurate and real-time blood pressure monitoring is often required. Systems that provide this type of blood pressure monitoring are known and include a catheter, usually inserted into an artery of the patient's circulatory system, a pressure sensor for measuring the arterial pressure, and a length of tubing between the catheter and the pressure sensor. To keep the tubing patent, so that the pressure in the tubing is approximately the pressure in the patient's artery, a fluid supply is coupled through the pressure sensor through another length of tubing so that the fluid supply is in fluid communication with the patient's circulatory system. In this way, the pressure sensor accurately and continuously reflects the pressure in the patient's artery.

It is also known that these blood pressure monitoring systems may also serve the purpose of taking periodic blood samples, thus eliminating the problems associated with multiple needle sticks. Thus, the blood pressure monitoring system may further include a sample site disposed in the tubing between the catheter and the pressure sensor from which blood may be withdrawn, a reservoir associated with the tubing upstream of the sample site, and a stopcock. To draw a blood sample, the stopcock is turned so as to shut off the flow of fluid from the fluid supply. Blood then flows from the patient along the tubing, through the sample site and towards or into the reservoir until there is whole blood in the sample site. A whole blood sample is then taken from the site. Any blood remaining in the tubing after the sample is taken may then be restored to the patient by reopening the stopcock and allowing fluid from the fluid supply to flow through the tubing toward the patient and to re-establish the monitoring of the patient's blood pressure.

In some such systems, the reservoir branches off of the tubing, such that any fluid collected therein is typically discarded, although in some situations the fluid may be restored toward the patient, such as where the reservoir is a syringe. In other systems, the reservoir is in-line with the tubing such that any fluid in the reservoir is always to be restored toward the patient. Such in-line reservoirs have inlet and outlet ports coupled into the tubing with a variable volume chamber therebetween. One example of an in-line reservoir is the piston-cylinder arrangement of U.S. Pat. No. 4,673,386.

In the device of that patent, the reservoir has a rigid wall comprised of a bottom and a side which define a cylinder having an oval cross section that receives an oval-shaped piston received through the upper opening of the cylinder so that the rigid face wall of the piston confronts the cylinder bottom to define a variable volume chamber therebetween. The piston is movable towards and away from the rigid bottom of the cylinder wall so as to increase and decrease the interior volume of the chamber. The piston has a minimum volume position with the rigid face of the piston adjacent the bottom of the cylinder at which the inlet and outlet ports remain in fluid communication through the chamber. As the piston is pulled away from the bottom, a negative pressure is created that pulls blood away from the patient, through the tubing, and toward the reservoir. Moving the piston back toward the bottom discharges fluid in the chamber back through the tubing and toward the patient. The in-line piston-cylinder device has certain drawbacks, however.

By way of example, a seal must be maintained between the piston and the cylinder side during movement of the piston. Such sealing members slide along the cylinder side and can be a source of potential leakage or contamination paths. Failure of the seal creates a leakage path for reservoir contents to escape to the environment. Further, as the piston is retracted to increase the reservoir volume, new surface area of the cylinder side is exposed to the reservoir contents. This provides a contamination path for bacteria, microbes, and other undesirable contaminants to enter the bloodstream. Conversely, when the piston is pushed in, the piston traverses the cylinder to expose a portion of the cylinder side to the external environment previously in contact with the reservoir contents. This also provides a leakage path for reservoir contents, such as contaminated blood, to escape to the environment.

Another shortcoming of in-line reservoirs is that they are active-pull devices in that pulling on the piston forcibly creates a negative pressure in the reservoir which strongly “pulls” blood away from the patient and toward the reservoir. In some cases, this pulling might create a sufficient pressure drop to collapse a patient's artery thereby preventing a blood sample and, more importantly, potentially harming the patient. Such pulling could also de-gas the blood potentially causing inaccurate blood gas values in the sample. Either could happen, for example, if the piston were pulled too quickly. The proper rate at which to retract the piston then becomes problematic, depending on such factors as the size of the artery and the age of the particular patient.

### SUMMARY OF THE INVENTION

The present invention provides an in-line reservoir which overcomes the above-mentioned drawbacks. To this end, and in accordance with the principles of the present invention, a flexible membrane is sealingly secured to the rigid wall so as to close off the reservoir opening such that there is no leakage path. The membrane flexes to vary the volume of the chamber defined between the rigid bottom wall of the reservoir and the underside of the membrane. The membrane thus has a minimum volume position where the membrane is closely adjacent the rigid wall to define a minimum volume of the chamber such that fluid may still flow between the inlet and outlet and through the chamber. The membrane is able to flex out of this minimum volume position to an expanded volume position. To hold the flexible membrane in the minimum volume position and/or to flex the membrane toward the rigid wall and away from an expanded volume position, a drive surface that engages the membrane is provided.

According to one aspect of the invention, the drive surface is coupled to the flexible membrane so that the membrane forcibly flexes away from the minimum volume position when the drive surface is moved away from the rigid wall. The forcible movement of the membrane creates a negative pressure that pulls blood and fluid from the tubing and patient toward the reservoir. The inherent give in the flexible membrane reduces the risk, however, of collapsing the lumen of a patient's artery or of de-gassing the patient's blood during the pull of the membrane.

In another aspect of the invention, the drive surface is uncoupled from the membrane but may engage a top surface of the membrane. In accordance with this other aspect, when the drive surface is moved away from the rigid wall, the membrane is free to flex away from the minimum volume position to an expanded volume position under fluid pressure such as caused by the blood pressure of the patient. Since the patient's blood pressure pumps blood and fluid into the reservoir, there is no risk of collapsing the patient's artery or of de-gassing the patient's blood.

By virtue of the foregoing, there is thus provided a diaphragm-based reservoir for use in a closed blood sampling system that eliminates potential leakage and contamination paths of prior in-line reservoirs and which further reduces or eliminates the risk of arterial collapse or blood de-gassing when taking a blood sample. These and other objects and advantages of the present invention shall be made apparent from the accompanying drawings and the description thereof.

### BRIEF DESCRIPTION OF THE DRAWINGS

The accompanying drawings, which are incorporated in and constitute a part of this specification, illustrate embodiments of the invention and, together with the general description of the invention given above and the detailed description of the embodiments given below, serve to explain the principles of the present invention.

FIG. 1 is a schematic cross-sectional view of a diaphragm-based reservoir in the minimum volume position in accordance with the principles of the present invention;

FIG. 2 is a schematic cross-sectional view of the diaphragm-based reservoir of FIG. 1 in an expanded volume position;

FIG. 3 is a cross-sectional view of a second embodiment of a diaphragm-based reservoir in the minimum volume position in accordance with the principles of the present invention;

FIG. 4 is a cross-sectional view of the diaphragm-based reservoir of FIG. 3 in an expanded volume position.

FIG. 5 is a cross-sectional view of the diaphragm-based reservoir of FIG. 3 taken along line 5-5;

FIG. 6 is a cross-sectional view of a third embodiment of a diaphragm-based reservoir similar to that shown in FIGS. 3-5, but showing the membrane and the drive surface being coupled;

FIG. 7 is a cross-sectional view of a fourth embodiment of a diaphragm-based reservoir similar to that shown in FIGS. 3-5, but showing an open channel formed in the surface of the rigid wall;

FIG. 8 is a cross-sectional view of the diaphragm-based reservoir of FIG. 7 taken along line 8-8 showing the channel in the rigid wall;

FIG. 9 is a cross-sectional view of a fifth embodiment of the diaphragm-based reservoir in the minimum volume position in accordance with the principles of the present invention showing the rigid wall having an oval bowl shape;

FIG. 10 is a cross-sectional view of the diaphragm-based reservoir of FIG. 9 taken along line 10-10;

FIG. 11 is a cross-sectional view of a sixth embodiment of the diaphragm-based reservoir in the minimum volume position in accordance with the principles of the present invention showing the rigid wall having a conical bowl shape;

FIG. 12 is a cross-sectional view of a seventh embodiment of a diaprhagm-based reservoir similar to that shown in FIG. 11, but showing a closed channel between inlet and outlet ports having an aperture to a chamber similar to that shown in FIG. 11.

FIG. 13 is a diagrammatic view of a closed blood sampling system incorporating a diaphragm-based reservoir in accordance with the principles of the present invention.

### DETAILED DESCRIPTION

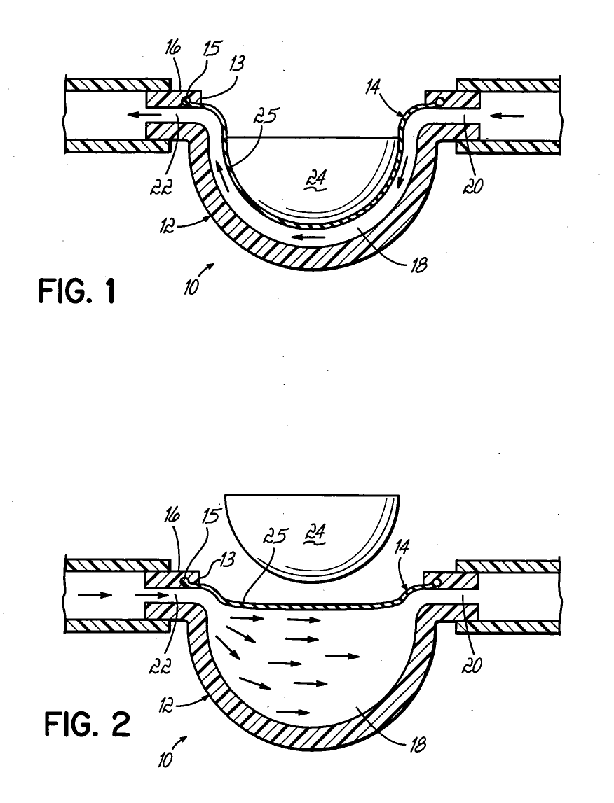

With reference to FIGS. 1-2, there is shown a simplified embodiment of a diaphragm-based reservoir 10 for a closed blood sampling system in accordance with the principles of the present invention. Reservoir 10 is defined by a rigid wall or lower housing 12 having an opening 13 and a flexible membrane 14 sealed to the opening periphery, such as by fixedly securing membrane 14 along its outer edge 15 to an upper edge 16 of rigid wall 12 to close off opening 13 and define an internal chamber 18. Reservoir 10 further includes fluid inlet and exit ports 20, 22 respectively in fluid communication with chamber 18 to allow fluid and/or blood to flow in or through the chamber. As shown in FIG. 1, the membrane 14 has a minimum volume position where the membrane 14 is spaced closely adjacent the rigid wall 12 to define a minimum volume of the chamber 18 such that fluid may still flow between inlet and exit ports 20, 22 and through chamber 18. To keep the membrane 14 in the minimum volume position, a drive surface 24, that is fluidly isolated from chamber 18, engages the top surface 25 of the membrane 14 and confines the membrane to the minimum volume position.

As shown in FIG. 2, when a blood sample is to be taken, the drive surface 24 is moved away from the rigid wall 12 so that the membrane 14 may flex out of its minimum volume position and to an expanded volume position as blood is caused to flow away from the patient and toward reservoir 10. Blood and other fluid in the line then flows back into chamber 18 through exit port 22. Advantageously, as the membrane 14 flexes, the outer edge 15 of the membrane 14 remains fixed and securely sealed to the upper edge 16 of rigid wall 12. After the whole blood sample is taken, the drive surface 24 is moved toward the rigid wall 12, flexing membrane 14 out of the expanded volume position and back toward its minimum volume position thereby discharging the chamber contents out through exit port 22 and back toward the patient. Because the seal between membrane 14 and rigid wall 12 is fixed during the expansion and contraction of chamber 18, there is no potential leakage path for blood to escape to the external environment, and there is no contamination path for bacteria and other contaminants to enter the bloodstream.

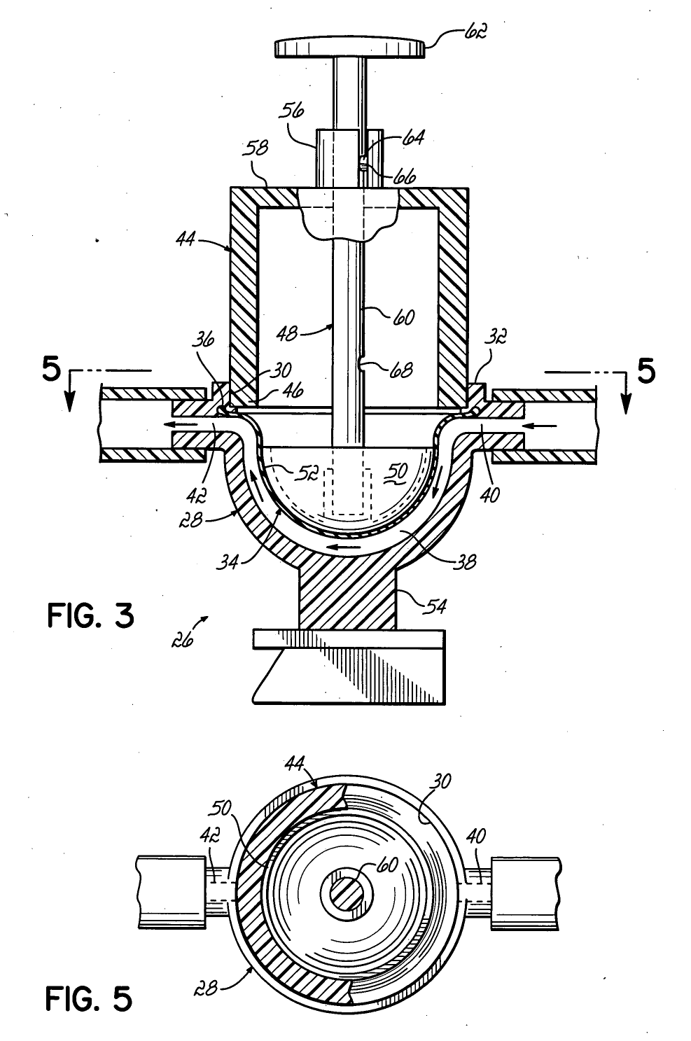

FIGS. 3-5 illustrate a second embodiment 26 of a diaphragm-based reservoir in accordance with the principles of the present invention. Reservoir 26 includes a lower housing 28 with an opening 30 and a flexible membrane 34 sealed to the opening periphery. Flexible membrane 34 is sealingly secured along its outer edge 36 to the upper edge 32 of the lower housing 28 to close off opening 30 and define a variable volume chamber 38. Adjacent the upper edge 32 of the lower housing 28 are inlet and exit ports 40, 42 respectively, in fluid communication with chamber 38 to allow fluid and/or blood to flow in or through the chamber. The reservoir 26 further includes an upper housing 44 having a lower edge 46 secured to the upper edge 32 of the lower housing 28. A plunger 48 extends through the upper housing 44 and couples to a drive surface 50 that engages an upper surface 52 of membrane 34.

In this embodiment, the lower housing 28 takes a circular bowl or hemispherical shape having a circular opening 30 along its top or upper edge 32. The lower housing 28 may further include a stem 54 adapted to cooperate with a mounting bracket to mount the reservoir 26 to a support structure (both not shown). The membrane 34 generally conforms to the shape of the lower housing 28 thus, in this embodiment, takes a circular bowl or hemispherical shape with a circular upper edge 36 that is sealed along the upper edge 32 of the lower housing 28. Since membrane 34 is conformed to the shape of the rigid wall 28, membrane 34 may be positioned adjacent the rigid wall 28, substantially in its minimum volume position, while in an unstretched or unflexed state. The upper housing 44 is generally cylindrical and includes a collar 56 extending from a top surface 58 of upper housing 44. The plunger 48 comprises a shaft 60 inserted through collar 56 and having one end coupled to a knob 62 external to the upper housing 44 that can be conveniently and easily manipulated by a healthcare provider to move the plunger 48. The opposed end of shaft 60 is coupled to the drive surface 50, located internal to the upper housing 44, to engage membrane 34. The drive surface 50 generally is conformed to the shape of the membrane 34 or lower housing 28 thus is hemispherically shaped and contacts the flexible membrane 34 substantially along its entire upper surface 52 when in the minimum volume position.

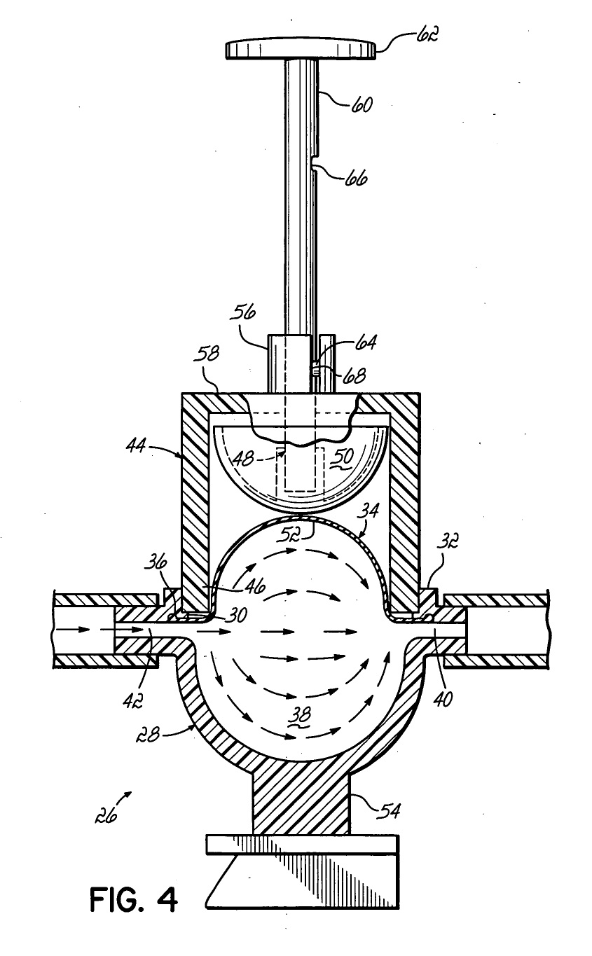

To secure the position of the drive surface 50 relative to the lower housing 28, collar 56 on upper housing 44 includes a detent 64. Plunger 48 further includes recesses 66, 68 along shaft 60. The upper housing 44 and the plunger 48 are operable such that when recesses 66, 68 engage detent 64, plunger 48 is fixedly secured to the upper housing 44 thereby preventing any movement of the drive surface 50 relative to the lower housing 28. As shown in FIG. 3, recess 66 is located along shaft 60 such that when recess 66 engages detent 64, the drive surface 50 engages the top surface 52 of the membrane 34 so as to be in the minimum volume position. Moreover, as shown in FIG. 4, recess 68 is located along shaft 60 such that when recess 68 engages detent 64, the drive surface 50 has been moved away from the lower housing 28 to define a maximum expanded volume position of membrane 34, with it being understood that all positions having a chamber volume greater than the minimum volume are expanded volume positions.

In the embodiment shown in FIGS. 3-5, membrane 34 is uncoupled from the drive surface 50. Because the membrane 34 and drive surface 50 are uncoupled, when the plunger 48 is moved away from the lower housing 28, the membrane 34 is able to flex away from the minimum volume position and to an expanded volume position under fluid pressure caused by the blood pressure of the patient. This passive expansion of the reservoir eliminates the risk of collapsing the lumen of the patient's artery and/or de-gassing of the patient's blood.

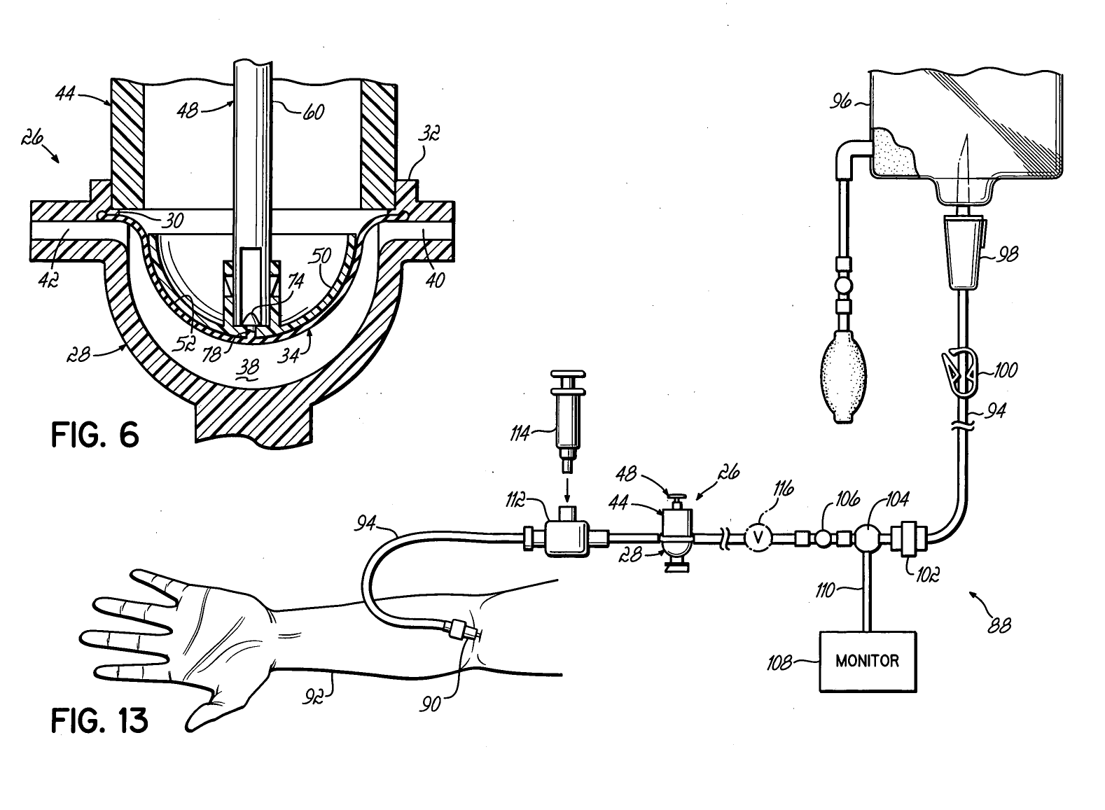

Alternatively, the membrane 34 and drive surface 50 may be coupled. To this end, a third embodiment of a diaphragm-based reservoir is modified from the embodiment of FIGS. 3-5 by the addition of a connecting structure as shown in FIG. 6 (in which like reference numerals refer to like features in FIGS. 3-5). The connecting structure includes a connecting member, such as a nipple 74, extending from the upper surface 52 of membrane 34, and a corresponding connecting member, such as aperture 78, associated with drive surface 50. Nipple 74 and aperture 78 cooperate to couple membrane 34 to the drive surface 50. In this way, as plunger 48 is moved away from the lower housing 28, the membrane 34 forcibly flexes away from the minimum volume position to create a negative pressure and pull blood from the patient and toward chamber 38. The inherent give in flexible membrane 38 reduces the risk of collapsing the lumen of the patient's artery and/or de-gassing of the patient's blood during the forcible flexing of the membrane.

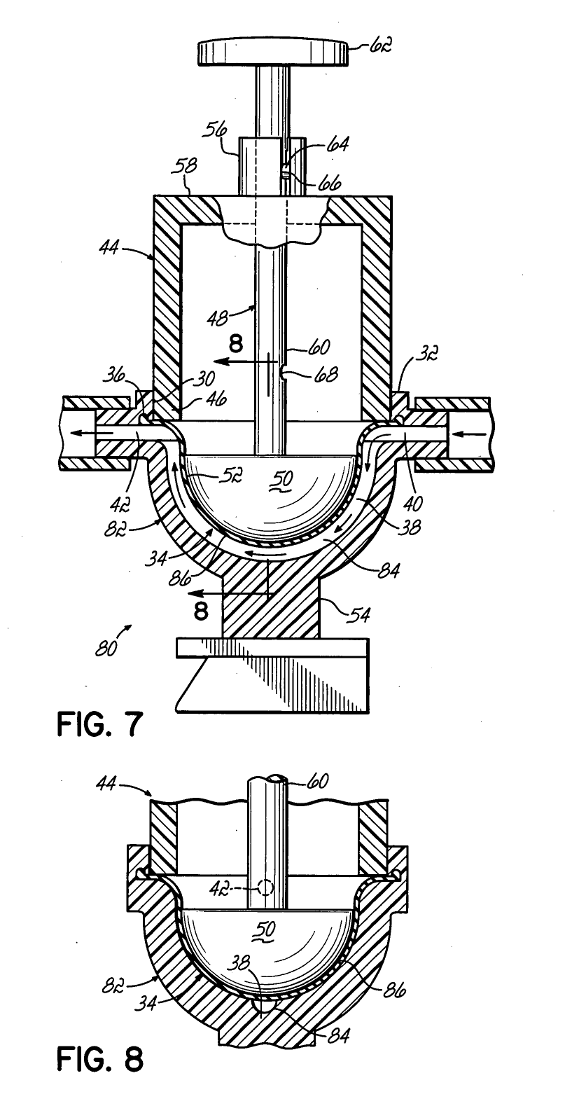

FIGS. 7-8, in which like reference numerals refer to like features in FIGS. 3-5, show a fourth embodiment 80 of a diaphragm-based reservoir in accordance with the principles of the present invention. Reservoir 80 includes a lower housing 82 having a channel 84 formed in lower housing 82. Channel 84 is in fluid communication with inlet port 40 and exit port 42 and comprises a portion of chamber 38. As shown more clearly in FIG. 8, channel 84 may be hemispherical or circular in cross-section, with an open top along a top surface of the lower housing 82. Advantageously, the diameter of channel 84 corresponds to the inner diameter of the tubing 94 (FIG. 13) with which reservoir 80 will be used. The flexible membrane 34 has a minimum volume position at which the lower surface 86 of membrane 34 engages lower housing 82 along a substantial portion of membrane 34. When in the minimum volume position, fluid may still flow between the inlet and outlet ports 40, 42 and through chamber 38, but mostly if not exclusively by way of channel 84. Thus in the minimum volume position, membrane 34 may be seen as a lid over channel 84 which closes off the top thereof. In this embodiment, when drive surface 50 is moved away from lower housing 82 to an expanded volume position, the chamber volume has a quick increase from the minimum volume in a stepwise manner as the larger surface areas of lower housing 82 and membrane surface 86 are exposed. Likewise, when drive surface 50 is moved toward the lower housing 82 to its minimum volume position, the chamber volume will quickly decrease in a stepwise manner as surface 86 engages lower housing 82.

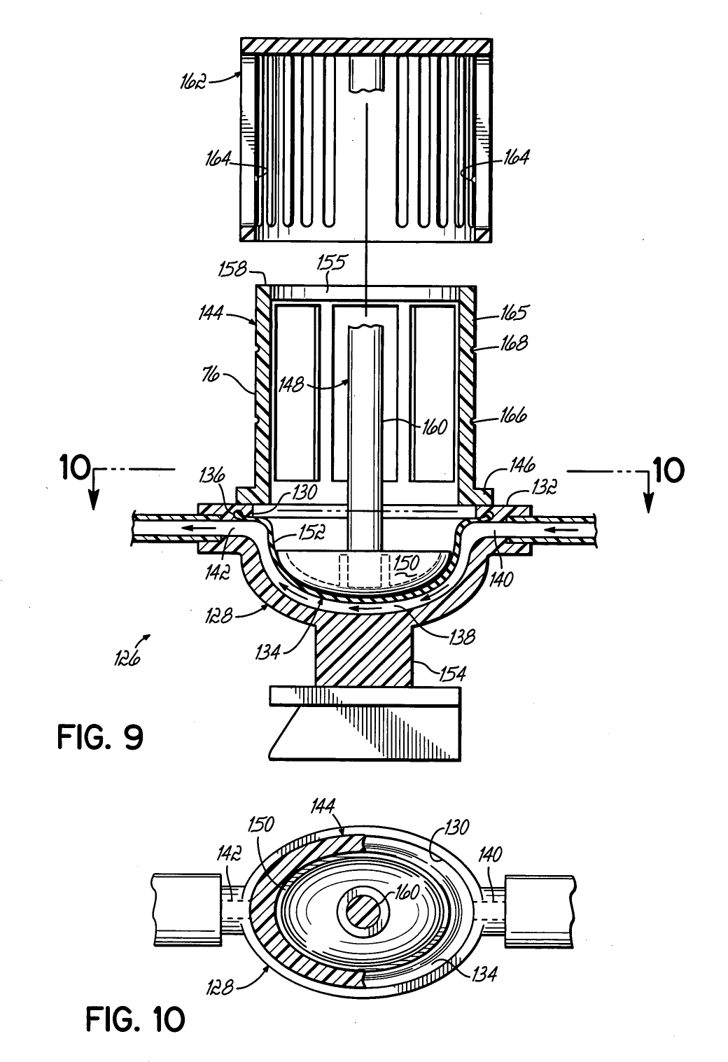

FIGS. 9-10 show a fifth embodiment 126 of a diaphragm-based reservoir in accordance with the principles of the present invention. Features that are similar to features in FIGS. 3-5 have been prefixed with a 1. Reservoir 126 includes a lower housing 128 with an opening 130 along its upper edge 132. A flexible membrane 134 is sealingly secured along its outer edge 136 to the upper edge 132 of the lower housing 128 to close off opening 130 and define a variable volume chamber 138. Adjacent the upper edge 132 of the lower housing 128 are inlet and exit ports 140, 142 respectively, in fluid communication with chamber 138 to allow fluid and/or blood to flow in or through the chamber. The reservoir 126 further includes an upper housing 144 having a lower edge 146 secured to the upper edge 132 of the lower housing 128. A plunger 148 extends through the upper housing 144 and couples to a drive surface 150 that engages an upper surface 152 of membrane 134.

As shown in FIG. 10, the lower housing 128 of the embodiment shown in FIG. 9 takes an elliptical or oval bowl shape having an elliptical opening 130 along its top edge 132. The lower housing may further include a stem 154 adapted to cooperate with a mounting bracket to mount the reservoir to a support structure (not shown). The membrane 134 takes an elliptical bowl shape with an elliptical upper edge 136 that is sealed along the upper edge 132 of the lower housing 128. Since membrane 134 is conformed to the shape of the rigid wall 128, membrane 134 may be positioned adjacent the rigid wall 128, substantially in its minimum volume position, while in an unstretched or unflexed state. The upper housing 144 is likewise elliptical and includes an open end 155 in the top surface 158 of the upper housing 144. The plunger 148 comprises a shaft 160 inserted through open end 155 and having one end coupled to a knob 162 external to the upper housing 144 that can be conveniently and easily manipulated by a healthcare provider to move the plunger 148. The knob 162 is elliptical and slightly larger than the upper housing 144 so that knob 162 slidingly engages the outer surface 165 of the housing 144 as knob 162 is moved away and toward the lower housing 128. The opposed end of shaft 160 is coupled to the drive surface 150 located internal to the upper housing 144 to engage membrane 134. The drive surface 150 has the elliptical bowl shape and contacts the flexible membrane 134 substantially along its entire upper surface 152 when in the minimum volume position.

As shown in FIG. 9, to secure the position of the drive surface 150 relative to the lower housing 128, knob 162 includes detent 164. Upper housing 144 further includes recesses 166 and 168. The upper housing 144 and knob 162 are operable such that when detent 164 engages recesses 166, 168, plunger 148 is fixedly secured to the upper housing 144 thereby preventing any movement of the drive surface 150 relative to the lower housing 128. Recess 166 is located along lower housing 144 such that when detent 164 engages recess 166, the drive surface 150 engages the top surface 152 of membrane 134 so as to be in the minimum volume position. Moreover, recess 168 is located along upper housing 144 such that when detent 164 engages recess 168, the drive surface 150 has been moved away from the lower housing 128 to define a maximum volume position of membrane 134. It should be appreciated that as with the embodiment in FIGS. 3-5, and as shown in FIG. 6, the embodiment in FIGS. 9-10 may be adapted to have the membrane and drive surface coupled in the same manner as that shown in FIG. 6. Additionally, the embodiment in FIGS. 9-10 may be further adapted to have a lower housing including a channel formed therein in the same manner as that shown in FIGS. 7-8.

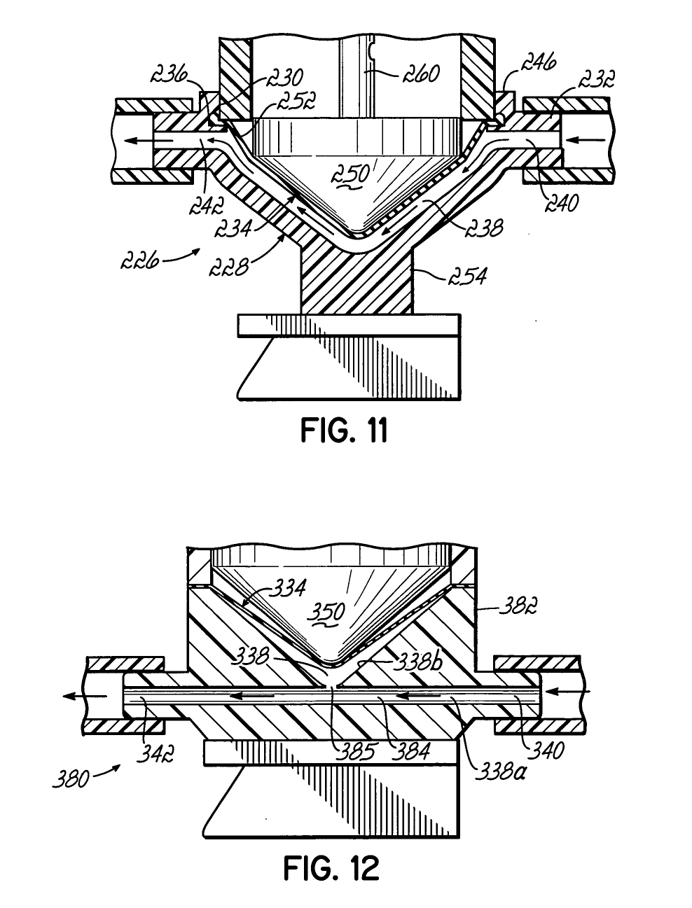

FIG. 11 shows a sixth embodiment 226 of a diaphragm-based reservoir in accordance with the principles of the present invention. Features that are similar to features in FIGS. 3-5 have been prefixed with a 2. As shown in FIG. 11, the lower housing 228 takes a conical bowl shape having a circular opening 230 along its top edge 232. The membrane 234 takes a conical bowl shape with a circular upper edge 236 that is sealed along the upper edge 232 of the lower housing 228. Since membrane 234 is conformed to the shape of the rigid wall 228, membrane 234 may be positioned adjacent the rigid wall 228, substantially in its minimum volume position, while in an unstretched or unflexed state. The drive surface 250 has the conical bowl shape and contacts the flexible membrane 234 substantially along its entire upper surface 252 when in the minimum volume position. It should be appreciated that as with the embodiment in FIGS. 3-5, and as shown in FIG. 6, the embodiment shown in FIG. 11 may be adapted to have the membrane and drive surface coupled in the same manner as that shown in FIG. 6. Additionally, the embodiment shown in FIG. 11 may be further adapted to have a lower housing including a channel formed therein in the same manner as that shown in FIGS. 7-8.

FIG. 12 shows a seventh embodiment 380 of a diaphragm-based reservoir in accordance with the principles of the present invention. Features that are similar to features in FIGS. 7-8 have been prefixed with a 3. Reservoir 380 includes a rigid wall 382 having a channel 384 formed in rigid wall 382. Channel 384 is in fluid communication with inlet port 340 and exit port 342 and comprises a portion of chamber 338, as identified by 338a. Unlike the open channel 84 formed in a surface of the surface of the rigid wall 82, as shown in FIGS. 7-8, channel 384 includes a portion completely enclosed by rigid wall 382 forming a closed channel. As shown in FIG. 12, channel 384 may be closed along a substantial portion of channel 384 so that it is thereby fluidly isolated from chamber 338. Channel 384, however, includes an access aperture 385 to provide fluid communication between channel 384 and the remaining portion 338b of chamber 338. Chamber portion 338b is similar to that previously described for the previous embodiments, except that chamber 338b is not directly coupled to inlet and outlet ports 340, 342, but solely communicates with inlet and outlet ports 340, 342 through channel 384 via aperture 385. The flexible membrane 334 has a minimum volume position at which the lower surface 386 of membrane 334 engages rigid wall 382 along a substantial portion of membrane 334. When in the minimum volume position, fluid may still flow between the inlet and outlet ports 340, 342 and through chamber 338 by way of channel 384. Advantageously, in the minimum volume position, aperture 385 is sealed off by membrane 334. When drive surface 350 is moved away from rigid wall 382 to an expanded volume position, the chamber volume has a quick increase from the minimum volume as aperture 385 is unsealed exposing chamber 338b to channel 384. Likewise, when drive surface 350 is moved toward the rigid wall 382 to its minimum volume position, the chamber volume will ultimately decrease rapidly.

FIG. 13 shows the diaphragm-based reservoir 26 in a closed blood sampling system 88. System 88 includes a catheter 90 for insertion into a patient's 92 blood vessel connected in a series via tubing 94 to a fluid supply 96. The fluid flows out of fluid supply 96 through conventional drip chamber 98. A clamp 100 may be mounted on tubing 94 adjacent drip chamber 98 in order to selectively block the flow of fluid from supply 96 with the patient 92. Downstream of the clamp 100 is a flush device 102, pressure transducer 104 and zeroing stopcock 106. Pressure transducer 104 is electrically connected to a monitor 108 by cable 110 for monitoring the patient's blood pressure. Downstream of stopcock 106 is the diaphragm-based reservoir 26 of the present invention. Immediately downstream of reservoir 26 is a sample site 112 that can be conveniently connected to syringe 114 for collecting a blood sample. The closed blood sampling system 88 may further include valve 116 coupled to the tubing 94 intermediate reservoir 26 and pressure transducer 104. Valve 116 is adapted to have on/off positions either allowing or preventing fluid flow through the valve.

To draw a blood sample in a sampling system 88 using the uncoupled diaphragm-based reservoir 26 shown in FIGS. 3-5, the flow of fluid through the reservoir 26 is stopped. This could be accomplished, for example, by the zeroing stopcock 106 or by valve 116. The plunger 48 is pulled away from the lower housing 28 so that flexible membrane 34 is unsupported and free to flex. The plunger 48 can be pulled away from the lower housing until recess 68 on shaft 60 engages the detent 64 of the upper housing 44 to define a maximum expanded volume position. Since membrane 34 is not supported by the drive surface 50, the patient's blood pressure pumps fluid downstream of reservoir 26 and blood from patient 92 through the tubing 86 and toward the reservoir 26 until whole blood is contained in the tubing 86 at sample site 112. A healthcare provider (not shown) then draws a whole blood sample in syringe 114 at sample site 112. After drawing the whole blood sample, the plunger 48 is then moved towards the lower housing 28. This movement flexes the membrane 34 towards the lower housing 28, thereby discharging the fluid and/or blood in reservoir 26 into tubing 86 and toward patient 84. The plunger 48 is pushed in until it reaches its minimum volume position and the recess 66 on shaft 60 engages the detent 64 of the upper housing 44. The flow of fluid from fluid supply 96 is then re-established to patient 92.

To draw a blood sample in a sampling system 88 using the coupled diaphragm-based reservoir 26 shown in FIG. 6, the flow of fluid through the reservoir may be stopped, for example, by the zeroing stopcock 106 or valve 116. Completely shutting off the flow, however, may be unnecessary in a coupled reservoir 26 as flush valve 102 provides some limitation on the flow from fluid supply 96, and forcibly flexing membrane 34 only pulls a small amount of fluid from the fluid supply and tubing upstream of the reservoir. The plunger 48 is pulled away from the lower housing 28, flexing the membrane 34 away from its minimum volume position. The plunger 48 can be pulled away from the lower housing until the recess 68 on shaft 60 engages the detent 64 of the upper housing 44 to define a maximum expanded volume position (see FIG. 4). This movement forcibly flexes the membrane 34 to create a negative pressure at the reservoir 26 thereby pulling blood away from patient 92 and toward reservoir 26. Blood and/or fluid flows by the sample site 112 and into the reservoir 26 until whole blood is contained in the tubing 94 at sample site 112. A healthcare provider (not shown) then draws a whole blood sample in syringe 114 at sample site 112. After drawing the whole blood sample, the plunger 48 is then moved towards the lower housing 28. This movement flexes membrane 34 towards the rigid wall 28, thereby discharging the fluid and/or blood in reservoir 26 into tubing 94 and toward patient 92. The plunger 48 is pushed in until it reaches its minimum volume position and the recess 66 on shaft 60 engages the detent 64 of the upper housing 44. The flow of fluid from fluid supply 96 is then re-established to patient 92 if the stopcock 106 or valve 116 was optionally used to stop the flow of fluid through the reservoir.

Using a closed blood sampling system incorporating a diaphragm-based reservoir provides a number of advantages. First reservoirs that use negative pressure to pull blood from the patient carry the risk that if the pressure is significantly reduced, the patient's artery may collapse and/or the patient's blood may de-gas. The coupled aspect of the present invention advantageously reduces such risks. Since the reservoir uses a flexible membrane to provide expanded reservoir volumes, this flexibility provides some additional “give” in the system by allowing the membrane to flex to accommodate large pressure changes rather than collapsing the patient's artery and/or de-gassing the blood. Moreover, the uncoupled aspect of this invention advantageously eliminates the risk of collapsing the patient's artery and/or of de-gassing the patient's blood. In this uncoupled aspect, there is no forcible expansion of the reservoir but rather the patient's blood pressure is what causes the membrane to flex to an expanded volume position. This effectively eliminates the risk of collapsing the lumen of the patient's artery and de-gassing of the patient's blood.

Another advantage of the present invention is that as the reservoir volume expands and collapses, the outer edge of the flexible membrane remains securely sealed to the lower housing, and so does not slide along either the lower or upper housing wall. Because the seal created at the edge of the flexible membrane and the rigid wall is fixed, there is no potential leak path for blood and other bio-hazardous fluids to escape to the environment. Moreover, there is also no contamination path for bacteria or other contaminants to enter the bloodstream.

While the present invention has been illustrated by the description of embodiments thereof, and while the embodiments have been described in considerable detail, it is not intended to restrict or in any way limit the scope of the appended claims to such detail. Additional advantages and modifications will readily appear to those skilled in the art. For example, in the embodiments shown herein, the membrane is described as being unstretched or unflexed when substantially in its minimum volume position. The membrane may, however, be unstretched in an expanded volume position and then stretched or flexed to be positioned into its minimum volume position. Such a membrane may be advantageous, such as for the uncoupled aspect of the present invention, in that as the drive surface moves away from the membrane, the membrane will have a tendency to return to its unflexed state, thereby providing an assist to pull fluid and/or blood into the reservoir. The membrane may be made from several types of materials that may depend on factors such as whether the membrane is formed so as to conform to the shape of the rigid wall. When the membrane is formed to correspond to the rigid wall, the membrane may advantageously be made from silicone, butyl, nitrile, urethane or other suitable materials having a low modulus that readily flex when acted upon. Moreover, some of these materials, such as silicone, may be further treated so as to reduce gas permeability. In the case the membrane does not conform to the rigid wall but comprises a flat sheet of material that is stretched or flexed when in the minimum volume position, the membrane may advantageously be made from natural or synthetic polyisoprene, EPDM, nitrile-EPDM blends or other suitable materials.

While the lower housing, membrane and drive surface has been described as having circular, oval and conical bowl shapes, a number of shapes may be used. Further, the rigid wall may not be a smooth, continuous surface, but may also be of multiple wall portions, such as the side and bottom of a cylinder, by way of example. Additionally, the lower housing may be made from a polycarbonate or acrylic material, but those having skill in the art will recognize other materials suitable for the lower housing.

In the embodiments shown herein, the membrane is sealed to the lower housing along the opening periphery so as to close off the opening to form the variable-volume chamber. It will be appreciated, however, that the membrane may be sealed to the lower housing at a location other than the opening periphery just as long as the opening is closed off and fluid flows between inlet and outlet ports when in the minimum volume position. For instance, if the opening periphery were spaced above the inlet and exit ports, then the membrane could be sealed to the lower housing rigid wall at a location above the inlet and exit ports yet below the opening periphery. The opening would still be closed off but fluid could flow between the inlet and outlet ports and through the chamber in the minimum volume position.

The reservoir may change depending on the particular set up and application. It is contemplated that the minimum volume of the chamber can be approximately 0.1 ml, although other minimum volumes may be appropriate depending on the flow dynamics of the system. Furthermore, in the maximum expanded volume position it is contemplated that the chamber will have a volume of approximately 12-13 ml. The maximum volume, however, will depend on such factors as the length and internal diameter of the tubing between the sample site 112 and the patient 92, and perhaps between the sample site 112 and the reservoir 26 (FIG. 13). The maximum volume must be large enough such that as fluid fills into the reservoir, enough fluid can flow through and past the sample site in order that a whole blood sample will be available at the sample site. Advantageously, some diluted blood will flow into the reservoir.

Moreover, while the closed blood sampling system has been described as part of an arterial pressure monitoring system, it is to be appreciated that the closed blood sampling system as described herein may be incorporated into other systems, such as venous infusion lines. The invention in its broader aspects is, therefore, not limited to the specific details, representative apparatus and method, and illustrative examples shown and described. Accordingly, departures may be made from such details without departing from the spirit or scope of the general inventive concept.

1. A reservoir for use in a closed blood sampling system, the reservoir comprising:
a rigid wall;
a flexible membrane overlying at least part of the rigid wall and sealingly secured thereto to define a variable volume chamber therebetween;
an inlet port and an exit port in fluid communication with the chamber;
the flexible membrane having a minimum volume position spaced closely adjacent the rigid wall to define a minimum volume at which fluid still flows between the inlet port and exit port through the chamber, the flexible membrane being able to flex out of the minimum volume position to an expanded volume position; and
a drive surface adapted to engage against the flexible membrane to hold the membrane in the minimum volume position.

2. The reservoir of claim 1, the rigid wall including a stem adapted to cooperate with a mounting bracket for mounting to a support.

3. The reservoir of claim 1, the rigid wall having a shape and the flexible membrane generally conforming to the shape of the rigid wall.

4. The reservoir of claim 1, the rigid wall having a shape and the drive surface generally conforming to the shape of the rigid wall.

5. The reservoir of claim 1, the rigid wall including a channel formed therein, with the channel having a portion thereof in fluid communication with the chamber in at least the expanded volume position.

6. The reservoir of claim 5, the channel being in fluid communication with the inlet port and the exit port.

7. The reservoir of claim 6, the channel being an open channel formed in a surface of the rigid wall.

8. The reservoir of claim 6, at least a portion of the channel being completely enclosed by the rigid wall.

9. The reservoir of claim 8, a substantial portion of the channel being completely enclosed by the rigid wall.

10. The reservoir of claim 1, the flexible membrane having a lower surface, the rigid wall engaging against at least a portion of the lower surface in the minimum volume position.

11. The reservoir of claim 1, the drive surface being fluidly isolated from the chamber.

12. The reservoir of claim 1, the flexible membrane having an upper surface, the drive surface engaging against substantially the entire upper surface when in the minimum volume position.

13. The reservoir of claim 1, the drive surface being positioned to move the membrane toward the rigid wall so as to reduce the volume of the chamber in a first direction of movement of the drive surface.

14. The reservoir of claim 13, the movement of the drive surface in the first direction causing a stepwise decrease in the volume of the chamber.

15. The reservoir of claim 1, the flexible membrane being able to flex away from the rigid wall to the expanded volume position in a second direction of movement of the drive surface.

16. The reservoir of claim 15, the movement of the drive surface in the second direction causing a stepwise increase in the volume of the chamber.

17. The reservoir of claim 1, the drive surface being coupled to the flexible membrane.

18. The reservoir of claim 17, the flexible membrane including a nipple.

19. The reservoir of claim 18, the drive surface adapted to couple to a nipple.

20. The reservoir of claim 1, the rigid wall being generally bowl shaped.

21. The reservoir of claim 20, the bowl shaped rigid wall being one of hemispherical, conical, and oval in shape.

22. The reservoir of claim 20, the rigid wall having an upper edge.

23. The reservoir of claim 20, the flexible membrane being generally bowl shaped.

24. The reservoir of claim 20, the drive surface being generally bowl shaped.

25. The reservoir of claim 22, the upper edge traversing at least one of a circular and elliptical path.

26. The reservoir of claim 22, the inlet and exit ports being adjacent the upper edge.

27. The reservoir of claim 23, the bowl shaped membrane being one of hemispherical, conical, and oval in shape.

28. The reservoir of claim 23, the flexible membrane having an upper edge.

29. The reservoir of claim 28, the upper edge traversing at least one of a circular and elliptical path.

30. The reservoir of claim 1 further comprising a plunger, the plunger comprising:
a shaft having a first and second end portion, the first end portion coupled to the drive surface; and
a knob coupled to the second end potion of the shaft for manipulation by a user.

31. The reservoir of claim 30 further comprising a housing overlying at least part of the flexible membrane and coupled to the rigid wall.

32. The reservoir of claim 31, at least one of the housing and plunger being adapted to fixedly secure the drive surface in the minimum volume position.

33. The reservoir of claim 31, at least one of the housing and plunger being adapted to fixedly secure the drive surface in a maximum expanded volume position.

34. The reservoir of claim 31, the housing having a lower edge.

35. The reservoir of claim 32, the housing further comprising a detent, the plunger further comprising a recess, the plunger fixedly securing the drive surface in the minimum volume position when the detent engages the recess.

36. The reservoir of claim 33, the housing further comprising a detent, the plunger further comprising a recess, the plunger fixedly securing the drive surface in a maximum expanded volume position when the detent engages the recess.

37. The reservoir of claim 34, the lower edge traversing at least one of a circular and elliptical path.

38. A closed blood sampling system comprising:
tubing adapted to be coupled between a fluid supply and a circulatory system of a patient;
a reservoir disposed in the tubing comprising:
a rigid wall;
a flexible membrane overlying at least part of the rigid wall and sealingly secured thereto to define a variable volume chamber therebetween;
an inlet port and an exit port in fluid communication with the chamber,
the flexible membrane having a minimum volume position spaced closely adjacent the rigid wall to define a minimum volume at which fluid still flows between the inlet port and exit port through the chamber, the flexible membrane being able to flex out of the minimum volume position to an expanded volume position; and
a drive surface adapted to engage against the flexible membrane to hold the membrane in the minimum volume position; and
a sampling site disposed in the tubing intermediate the patient and the reservoir adapted to draw blood from the tubing.

39. The closed blood sampling system of claim 38, the rigid wall having a shape and the flexible membrane generally conforming to the shape of the rigid wall.

40. The closed blood sampling system of claim 38, the rigid wall having a shape and the drive surface generally conforming to the shape of the rigid wall.

41. The closed blood sampling system of claim 38, the rigid wall including a channel formed therein, with the channel having a portion thereof in fluid communication with the chamber in at least the expanded volume position.

42. The closed blood sampling system of claim 41, the channel being in fluid communication with the inlet port and the exit port.

43. The closed blood sampling system of claim 42, the channel being an open channel formed in a surface of the rigid wall.

44. The closed blood sampling system of claim 42, at least a portion of the channel being completely enclosed by the rigid wall.

45. The closed blood sampling system of claim 44, a substantial portion of the channel being completely enclosed by the rigid wall.

46. The closed blood sampling system of claim 38, the flexible membrane having a lower surface, the rigid wall engaging against at least a portion of the lower surface in the minimum volume position.

47. The closed blood sampling system of claim 38, the drive surface being fluidly isolated from the chamber.

48. The closed blood sampling system of claim 38, the flexible membrane having an upper surface, the drive surface engaging against substantially the entire upper surface when in the minimum volume position.

49. The closed blood sampling system of claim 38, the drive surface being positioned to move the membrane toward the rigid wall so as to reduce the volume of the chamber in a first direction of movement of the drive surface.

50. The closed blood sampling system of claim 38, the flexible membrane being able to flex away from the rigid wall to the expanded volume position in a second direction of movement of the drive surface.

51. The closed blood sampling system of claim 38, the drive surface being coupled to the flexible membrane.

52. The closed blood sampling system of claim 38 further comprising a plunger, the plunger comprising:
a shaft having a first and second end portion, the first end portion coupled to the drive surface; and
a knob coupled to the second end potion of the shaft for manipulation by a user.

53. The closed blood sampling system of claim 52 further comprising a housing overlying at least part of the flexible membrane and coupled to the rigid wall.

54. The closed blood sampling system of claim 53, at least one of the housing and plunger being adapted to fixedly secure the drive surface in the minimum volume position.

55. The closed blood sampling system of claim 53, at least one of the housing and plunger being adapted to fixedly secure the drive surface in a maximum expanded volume position.

56. The closed blood sampling system of claim 54, the housing further comprising a detent, the plunger further comprising a recess, the plunger fixedly securing the drive surface in the minimum volume position when the detent engages the recess.

57. The closed blood sampling system of claim 55, the housing further comprising a detent, the plunger further comprising a recess, the plunger fixedly securing the drive surface in a maximum expanded volume position when the detent engages the recess.

58. A method of sampling blood in a closed blood sampling system wherein the sampling system has tubing intermediate a fluid supply and the circulatory system of a patient, a reservoir disposed in the tubing having a rigid wall, a flexible membrane overlying at least part of the rigid wall and sealingly secured thereto to define a variable volume chamber therebetween, an inlet port and an exit port in fluid communication with the chamber, the flexible membrane with a minimum volume position spaced closely adjacent the rigid wall to define a minimum volume at which fluid still flows between the inlet port and exit port through the chamber, the flexible membrane being able to flex out of the minimum volume position to an expanded volume position, a drive surface adapted to engage against the flexible membrane to hold the membrane in the minimum volume position, and a sampling site disposed in the tubing intermediate the patient and the reservoir adapted to draw blood from the tubing, the method comprising:
flexing the membrane away from the minimum volume position to an expanded volume position so as to provide whole blood at the sampling site;
drawing whole blood from the sampling site; and,
flexing the membrane toward the minimum volume position so as to discharge fluid from the reservoir toward the patient.

59. The method of claim 58 further comprising attaching the reservoir to a mounting bracket to mount the reservoir to a support.

60. The method of claim 58 wherein flexing the membrane away from the minimum volume position comprises forcibly flexing the membrane away from the minimum volume position.

61. The method of claim 60 wherein forcibly flexing the membrane away from the minimum volume position comprises:
coupling the drive surface to the flexible membrane; and
moving the drive surface in a second direction of movement away from the rigid wall.

62. The method of claim 58 wherein flexing the membrane toward the minimum volume position comprises forcibly flexing the membrane toward the minimum volume position.

63. The method of claim 62 wherein forcibly flexing the membrane toward the minimum volume position comprises moving the drive surface in a first direction of movement toward the rigid wall.

64. The method of claim 58 further comprising:
disposing a valve intermediate the fluid supply and the reservoir; and
closing the valve prior to flexing the membrane away from the minimum volume position to an expanded volume position.

65. The method of claim 64 wherein flexing the membrane away from the minimum volume position comprises flexing the membrane using fluid pressure of the patient's circulatory system.

66. The method of claim 58 wherein flexing the membrane toward the minimum volume position includes flexing the membrane into contact with a portion of the rigid wall.

67. A reservoir for use in a closed blood sampling system, the reservoir comprising:
a lower housing having a rigid wall with an opening;
a flexible membrane sealingly secured to the lower housing and closing off the opening to define a variable volume chamber therebetween;
an inlet port and an exit port in fluid communication with the chamber;
an upper housing coupled to the lower housing;
the flexible membrane having a minimum volume position spaced closely adjacent the rigid wall to define a minimum volume at which fluid still flows between the inlet port and exit port though the chamber, the flexible membrane being able to flex out of the minimum volume position to an expanded volume position; and,
a moveable plunger having a first portion received through the upper housing and having a drive surface fluidly isolated from the chamber, the drive surface being coupled to the flexible membrane, and a second portion for manipulation by a user, such that movement of the plunger in a first direction forcibly flexes the membrane toward the expanded volume position to draw fluid from a patient toward the reservoir so as to provide whole blood at a sampling site intermediate the patient and reservoir, and movement of the plunger in a second direction flexes the membrane toward the minimum volume position to discharge fluid from the reservoir toward the patient.

68. The reservoir of claim 67, the rigid wall including a stem adapted to cooperate with a mounting bracket for mounting to a support.

69. The reservoir of claim 67, the rigid wall having a shape and the flexible membrane generally conforming to the shape of the rigid wall.

70. The reservoir of claim 67, the rigid wall having a shape and the drive surface generally conforming to the shape of the rigid wall.

71. The reservoir of claim 67, the rigid wall including a channel formed therein, with the channel having a portion thereof in fluid communication with the chamber in at least the expanded volume position.

72. The reservoir of claim 71, the channel being in fluid communication with the inlet port and the exit port.

73. The reservoir of claim 72, the channel being an open channel formed in a surface of the rigid wall.

74. The reservoir of claim 72, at least a portion of the channel being completely enclosed by the rigid wall.

75. The reservoir of claim 74, a substantial portion of the channel being completely enclosed by the rigid wall.

76. The reservoir of claim 67, the flexible membrane having a lower surface, the rigid wall engaging against at least a portion of the lower surface in the minimum volume position.

77. The reservoir of claim 67, the flexible membrane having an upper surface, the drive surface engaging against substantially the entire upper surface when in the minimum volume position.

78. The reservoir of claim 67, the flexible membrane including a nipple.

79. The reservoir of claim 78, the drive surface adapted to couple to a nipple.

80. The reservoir of claim 67, at least one of the housing and plunger being adapted to fixedly secure the drive surface in the minimum volume position.

81. The reservoir of claim 67, at least one of the housing and plunger being adapted to fixedly secure the drive surface in a maximum expanded volume position.

82. A reservoir for use in a closed blood sampling system, the reservoir comprising:
a lower housing having a rigid wall with an opening;
a flexible membrane sealingly secured to the lower housing and closing off the opening to define a variable volume chamber therebetween;
an inlet port and an exit port in fluid communication with the chamber;
an upper housing coupled to the lower housing;
the flexible membrane having a minimum volume position spaced closely adjacent the rigid wall to define a minimum volume at which fluid still flows between the inlet port and exit port through the chamber, the flexible membrane being able to flex out of the minimum volume position to an expanded volume position; and
a moveable plunger having a first portion received through the upper housing and having a drive surface fluidly isolated from the chamber, and a second portion for manipulation by a user, such that movement of the plunger in a first direction flexes the membrane toward the expanded volume position under fluid pressure of a patient's circulatory system so as to provide whole blood at a sampling site intermediate the patient and reservoir, and movement of the plunger in a second direction flexes the membrane toward the minimum volume position to discharge fluid from the reservoir toward the patient.

83. The reservoir of claim 82, the rigid wall including a stem adapted to cooperate with a mounting bracket for mounting to a support.

84. The reservoir of claim 82, the rigid wall having a shape and the flexible membrane generally conforming to the shape of the rigid wall.

85. The reservoir of claim 82, the rigid wall having a shape and the drive surface generally conforming to the shape of the rigid wall.

86. The reservoir of claim 82, the rigid wall including a channel formed therein, with the channel having a portion thereof in fluid communication with the chamber in at least the expanded volume position.

87. The reservoir of claim 86, the channel being in fluid communication with the inlet port and the exit port.

88. The reservoir of claim 87, the channel being an open channel formed in a surface of the rigid wall.

89. The reservoir of claim 87, at least a portion of the channel being completely enclosed by the rigid wall.

90. The reservoir of claim 89, a substantial portion of the channel being completely enclosed by the rigid wall.

91. The reservoir of claim 82, the flexible membrane having a lower surface, the rigid wall engaging against at least a portion of the lower surface in the minimum volume position.

92. The reservoir of claim 82, the flexible membrane having an upper surface, the drive surface engaging against substantially the entire upper surface when in the minimum volume position.

93. The reservoir of claim 82, at least one of the housing and plunger being adapted to fixedly secure the drive surface in the minimum volume position.

94. The reservoir of claim 82, at least one of the housing and plunger being adapted to fixedly secure the drive surface in a maximum expanded volume position.

95. A reservoir for use in a closed blood sampling system, the reservoir comprising:
a lower housing having a rigid wall with an opening;
a flexible membrane sealingly secured to the lower housing and closing off the opening to define a variable volume chamber therebetween;
a channel formed in the rigid wall having an inlet port and an exit port, the channel in fluid communication with the chamber along at least a portion of the channel;
the flexible membrane having a minimum volume position with at least a portion of a lower surface of the membrane engaging the rigid wall to define a minimum volume at which fluid still flows between the inlet port and exit port through the chamber, the flexible membrane being able to flex out of the minimum volume position to an expanded volume position; and
a drive surface adapted to engage against the flexible membrane to hold the membrane in the minimum volume position.

96. The reservoir of claim 95, the channel being an open channel formed in a surface of the rigid wall.

97. The reservoir of claim 95, at least a portion of the channel being completely enclosed by the rigid wall.

98. The reservoir of claim 97, a substantial portion of the channel being completely enclosed by the rigid wall.

99. The reservoir of claim 95, the drive surface being positioned to move the membrane toward the rigid wall so as to reduce the volume of the chamber in a first direction of movement of the drive surface.

100. The reservoir of claim 95, the flexible membrane being able to flex away from the rigid wall to the expanded volume position in a second direction of movement of the drive surface.

101. The reservoir of claim 95, the drive surface being coupled to the flexible membrane.

102. The reservoir of claim 95 further comprising a plunger, the plunger comprising:
a shaft having a first and second end portion, the first end portion coupled to the drive surface; and
a knob coupled to the second end portion of the shaft for manipulation by a user.

103. A reservoir for use in a closed blood sampling system, the reservoir comprising:
a lower housing having a rigid wall with an opening;
a flexible membrane sealingly secured to the lower housing and closing off the opening to define a variable volume chamber therebetween;
an inlet port and an exit port;
a channel in a surface of the rigid wall and extending between the inlet port and exit port, the channel being exposed to the chamber through an open top of the channel;
the flexible membrane having a minimum volume position closing off the open top of the channel without interfering flow between the inlet port and exit port through the channel, the flexible membrane being able to flex out of the minimum volume position to an expanded volume position; and
a drive surface adapted to engage against the flexible membrane to hold the membrane in the minimum volume position.

104. The reservoir of claim 103, the drive surface being positioned to move the membrane toward the channel so as to reduce the volume of the chamber in a first direction of movement of the drive surface.

105. The reservoir of claim 103, the flexible membrane being able to flex away from the channel to the expanded volume position in a second direction of movement of the drive surface.

106. The reservoir of claim 103, the drive surface being coupled to the flexible membrane.

107. The reservoir of claim 103 further comprising a plunger, the plunger comprising:
a shaft having a first and second end portion, the first end portion coupled to the drive surface; and
a knob coupled to the second end portion of the shaft for manipulation by a user.

108. A reservoir for use in a closed blood sampling system, the reservoir comprising:
a lower housing having a rigid wall with an opening;
a flexible membrane sealingly secured to the lower housing and closing off the opening to define a variable volume chamber therebetween;
an inlet port and an exit port;
a channel extending through the lower housing between the inlet port and exit port in fluid isolation from the chamber;
an access aperture in the rigid wall fluidly communicating between the channel and the chamber;
the flexible membrane having a minimum volume position sealing off the access aperture, the flexible membrane being able to flex out of the minimum volume position to an expanded volume position thereby unsealing the access aperture; and
a drive surface adapted to engage against the flexible membrane to hold the membrane in the minimum volume position.

109. The reservoir of claim 108, the drive surface being positioned to move the membrane toward the access aperture so as to reduce the volume of the chamber in a first direction of movement of the drive surface.

110. The reservoir of claim 108, the flexible membrane being able to flex away from the access aperture to the expanded volume position in a second direction of movement of the drive surface.

111. The reservoir of claim 108, the drive surface being coupled to the flexible membrane.

112. The reservoir of claim 108 further comprising a plunger, the plunger comprising:
a shaft having a first and second end portion, the first end portion coupled to the drive surface; and
a knob coupled to the second end portion of the shaft for manipulation by a user.

113. A reservoir for use in a closed blood sampling system, the reservoir comprising:
a rigid wall;
a flexible membrane overlying at least part of the rigid wall and sealingly secured relative thereto to define a variable volume chamber therebetween;
an inlet port and an exit port in fluid communication with the chamber;
the flexible membrane having a minimum volume position spaced closely adjacent the rigid wall to define a minimum volume at which fluid still flows between the inlet port and exit port through the chamber, the flexible membrane being able to flex out of the minimum volume position to an expanded volume position; and
a drive surface adapted to engage against the flexible membrane to hold the membrane in the minimum volume position.

114. A method of sampling blood in a closed blood sampling system wherein the sampling system has tubing intermediate a fluid supply and the circulatory system of a patient, a reservoir disposed in the tubing having a rigid wall, a flexible membrane overlying at least part of the rigid wall and sealingly secured relative thereto to define a variable volume chamber therebetween, an inlet port and an exit port in fluid communication with the chamber, the flexible membrane with a minimum volume position spaced closely adjacent the rigid wall to define a minimum volume at which fluid still flows between the inlet port and exit port through the chamber, the flexible membrane being able to flex out of the minimum volume position to an expanded volume position, a drive surface adapted to engage against the flexible membrane to hold the membrane in the minimum volume position, and a sampling site disposed in the tubing intermediate the patient and the reservoir adapted to draw blood from the tubing, the method comprising:
flexing the membrane away from the minimum volume position to an expanded volume position so as to provide whole blood at the sampling site;
drawing whole blood from the sampling site; and,
flexing the membrane toward the minimum volume position so as to discharge fluid from the reservoir toward the patient.
